(19) United States
(12) Patent Application Publication
(10) Pub. No.: US 2023/0013879 A1
(43) Pub. Date: Jan. 19, 2023

(54) GROUNDING CUFF SYSTEM

(71) Applicant: Advanced Neuromodulation Systems, Inc., Plano, TX (US)

(72) Inventors: Mary Khun Hor-Lao, Prosper, TX (US); Binesh Balasingh, Prosper, TX (US); Scott DeBates, Frisco, TX (US)

(21) Appl. No.: 17/377,299

(22) Filed: Jul. 15, 2021

(51) Int. Cl. A61B 18/16; A61B 5/0205; A61B 5/021

(52) U.S. Cl. CPC A61B 18/16; A61B 5/02055; A61B 5/021; A61B 2018/00577

(57) ABSTRACT

Embodiments of the present disclosure provide a grounding cuff having a flexible body and an inflatable bladder disposed within the flexible body. The inflatable body is configurable to an inflated state and a deflated state. In the inflated stated, the inflatable bladder applies pressure to the grounding cuff to provide and maintain improved contact between the ground cuff and skin of a patient. Additionally, the inflatable bladder includes an inlet configured to receive a fluid from a hose, the inflatable bladder configured to the inflated state based on the fluid received via the hose. The grounding cuff may include a grounding pad coupled to a ground terminal to extract energy from the patient during a medical therapy, such as ablation therapy. The inflatable bladder may be configured to the inflated state to promote strong contact between the grounding pad and skin of the patient.

### TECHNICAL FIELD

Aspects of the present disclosure relate generally to ablation therapy systems and, more particularly, to devices providing improved grounding during treatment of a patient during ablation therapy.

### BACKGROUND

High frequency ablation, such as radio frequency (RF) ablation, is a medical therapy that is employed to treat a number of patient disorders. For example, RF frequency nerve ablation may be used to treat osteoarthritic pain of the spine through the destruction of nerves using RF energy. In an RF ablation system, an RF generator provides RF energy to one or more electrodes to ablate neural tissue. A cannula may be used to guide and position the electrode(s) proximate to a target site within a patient's body prior to delivery of the RF energy. In addition to the RF generator, RF ablation devices and systems may include various components, such as power electronics, peripheral devices including user interfaces, cooling systems, and the like.

To safely deliver high frequency ablation therapy, such as RF ablation therapy, a grounding pad may be secured to a patient to close an electrical circuit between the patient and the RF ablation device. A grounding pad may be secured to a patient while the patient receives RF ablation therapy to remove electrical energy delivered to the patient by the RF ablation device. If adequate contact between the grounding pad and the patient's skin is not maintained during delivery of RF ablation therapy, heat may be generated that could cause discomfort to the patient or, in severe instances, burns at the site of the grounding pad. Present techniques for securing grounding pads to patients involve the use of adhesives, but such techniques have proven ineffective with respect to maintaining adequate contact between the grounding pad and the patient. For example, during a therapy session, sweat from the patient may cause portions of the grounding pad to separate from the skin, resulting in discomfort or even burns. To improve the contact of the grounding pad to the patient, the area at which the grounding pad is to be placed may be shaved to remove hair that may hinder retention of the grounding pad to the patient's skin by the adhesive. However, instances have occurred in which shaving was not performed or performed inadequately, resulting in inadequate contact between the grounding pad and the patient's skin.

### SUMMARY

The present application is directed to a system that includes a grounding cuff having a grounding pad configured to maintain improved electrical contact to skin of a patient so that, during performance of a high frequency ablation procedure (e.g., RF ablation), a closed electrical circuit is maintained to allow efficient removal of electrical energy associated with the therapy while minimizing the risk of discomfort or other harmful conditions. The grounding cuff may include a flexible body having an inflatable bladder disposed therein. The inflatable bladder may be configured to be in an inflated stated and a deflated state. The inflatable bladder may include an inlet configured to receive a fluid (e.g., air) to transition the inflatable bladder from a deflated state to an inflated state. When in the inflated state, the inflatable bladder may apply pressure to the grounding pad to establish consistent contact between the grounding pad and the skin of the patient without requiring the use of adhesives or shaving.

The grounding pad may be formed from an electrically conductive material that is configured to contact a patient's skin and extract electrical energy from the patient during ablation therapy. Additionally, the grounding cuff includes an electrical connector configured to couple the grounding pad to a first ground terminal, which may be distinct from a ground terminal used to provide grounding of a high frequency ablation device, such as an RF ablation device.

In an additional aspect of the disclosure, a method of manufacturing a grounding cuff is disclosed. The method may include obtaining a flexible body. Additionally, the method may include disposing an inflatable bladder within the flexible body. The inflatable bladder may include an inlet configured to receive fluid. Further, the method may include coupling a grounding pad to the flexible body. The grounding pad may be formed from an electrically conductive material having at least a first surface that is disposed external to the flexible body and that is configured to contact skin of a patient. Additionally, the method may include coupling an electrical connector to the grounding pad. The electrical connector may be configured to couple the grounding pad to a first ground terminal.

In yet another aspect of the disclosure, a system is disclosed that may include a high frequency ablation device and a grounding cuff. The high frequency ablation device may include at least one electrode coupled to a high frequency generator of the high frequency ablation device. The at least one electrode may be configured to deliver high frequency ablation therapy to a patient. The grounding cuff may include a grounding pad configured to maintain improved electrical contact to skin of a patient so that, during performance of a high frequency ablation procedure (e.g., RF ablation), a closed electrical circuit is maintained to allow efficient removal of electrical energy associated with the therapy while minimizing the risk of discomfort or other harmful conditions. The grounding cuff may include a flexible body having an inflatable bladder disposed therein. The inflatable bladder may be configured to be in an inflated state and a deflated state. The inflatable bladder may include an inlet configured to receive a fluid (e.g., air) to transition the inflatable bladder from a deflated state to an inflated state. When in the inflated state, the inflatable bladder may apply pressure to the grounding pad so as to establish consistent contact between the grounding pad and the skin of the patient without requiring the use of adhesives or shaving.

The foregoing has outlined rather broadly the features and technical advantages of the present disclosure in order that the detailed description of the disclosure that follows may be better understood. Additional features and advantages of the disclosure will be described hereinafter which form the subject of the claims of the disclosure. It should be appreciated by those skilled in the art that the conception and specific embodiment disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings, in which:

FIG. 1 is a block diagram illustrating aspects of a grounding cuff system according to embodiments of the present disclosure;

FIG. 2 is a block diagram illustrating aspects of a grounding cuff according to embodiments of the present disclosure;

FIG. 3 is a block diagram illustrating aspects of a grounding cuff according to embodiments of the present disclosure;

FIG. 4 is a block diagram illustrating aspects of a grounding pad according to embodiments of the present disclosure;

FIG. 5 is a flow diagram of a method for providing high frequency ablation therapy using a grounding cuff according to embodiments of the present disclosure;

FIG. 6 is a flow diagram of a method for manufacturing a ground cuff according to embodiments of the present disclosure; and

FIG. 7 is a block diagram illustrating additional aspects of a grounding cuff according to embodiments of the present disclosure.

It should be understood that the drawings are not necessarily to scale and that the disclosed aspects are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular aspects illustrated herein.

### DETAILED DESCRIPTION

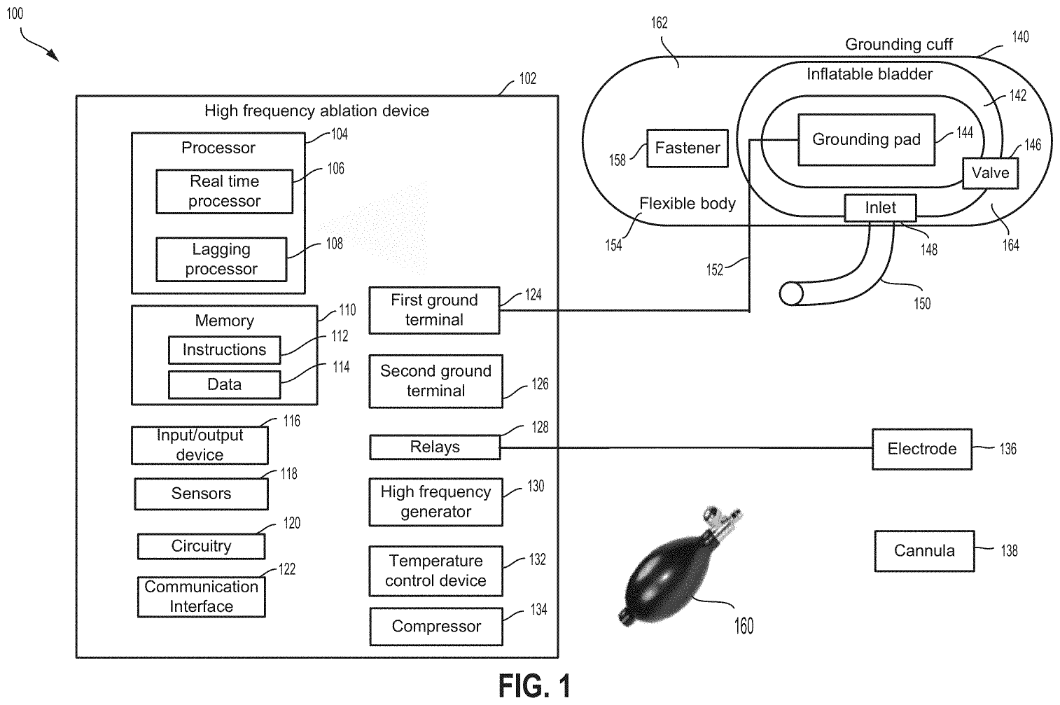

Various features and advantageous details are explained more fully with reference to the non-limiting embodiments that are illustrated in the accompanying drawings and detailed in the following description. Descriptions of well-known starting materials, processing techniques, components, and equipment are omitted so as not to unnecessarily obscure the disclosure in detail. It should be understood, however, that the detailed description and the specific examples, while indicating embodiments of the disclosure, are given by way of illustration only, and not by way of limitation. Various substitutions, modifications, additions, and/or rearrangements within the spirit and/or scope of the underlying inventive concept will become apparent to those skilled in the art from this disclosure. It is noted that FIGS. 1-6 use like reference numbers to represent the same or similar components except where otherwise specified.

Referring to FIG. 1, a block diagram illustrating aspects of a grounding cuff system according to embodiments of the present disclosure is shown as a system 100. As shown in FIG. 1, system 100 includes high frequency ablation device 102 (e.g., a radio frequency (RF) ablation device) and grounding cuff 140. As illustrated in FIG. 1, grounding cuff 140 may include flexible body 154, fastener 158, inflatable bladder 142, grounding pad 144, inlet 148, valve 146, and electrical connector 152. Flexible body 154 may be formed from a flexible fabric (e.g., a polymeric fabric such as nylon or cotton) to allow grounding cuff 140 to be wrapped around a portion of the body of the patient. For example, first end 162 may be placed on the patient's body and the second end may be wrapped around the patient's body (e.g., a leg, an arm, an abdomen, etc.) such that grounding pad 144 is in contact with skin of the patient. Grounding cuff 140 may be secured a desired position via fastener 158. Fastener 158 may be any of Velcro™, a buckle, or other fastening device. Fastener 158 may be configured to attach and hold first end 162 to second end 164. In some aspects, a fastener may also be disposed on second end 164. For example, where fastener 158 is Velcro™, a hook portion of the Velcro™ may be disposed on first end 162 and a loop portion of the Velcro™ may be disposed on second end 164 such that the hook portion and the loop portion may be releasably coupled once flexible body 154 is wrapped around a portion of the body of the patient.

Inflatable bladder 142 may be disposed within flexible body 154. Inflatable bladder 142 may be formed from any material (e.g., rubber, plastic, or other material) capable of receiving and holding a fluid. Inflatable bladder 142 may be configurable to an inflated state or a deflated state. For example, as fluid is introduced into inflatable bladder 142, inflatable bladder may transition to the inflated state and as fluid is released from inflatable bladder 142, inflatable bladder may transition from the inflated state to the deflated state. As inflatable bladder 142 is filled with a fluid (e.g., air) it may expand (i.e., transition to the inflated state), which may apply pressure to grounding pad 144, thereby forcing the first surface of the grounding pad 144 to contact the skin of the patient.

To facilitate introduction of the fluid into inflatable bladder 142, inlet 148 may be provided. Inlet 148 may be configured to attach a mechanism adapted to deliver fluid to inflatable bladder 142, such as by hose 150. For example, inlet 148 may attach to the mechanism adapted to deliver fluid, such as to hose 150, through a threaded connector, a compression fit, a friction fit, or another attachment technique. Hose 150 may be formed from a flexible polymeric material (e.g., rubber, plastic, etc.) suitable for delivery of fluid to inflatable bladder 142 via inlet 148. In some aspects, hose 150 may be configured to attach to a fluid introduction device, such as compressor 134 or hand pump 160. Additionally, inflatable bladder 142 may include valve 146 (e.g., a mechanical valve) configured to release the fluid retained in inflatable bladder 142 to transition inflatable bladder 142 from the inflated stated to the deflated state.

Grounding pad 144 may be formed from any electrically conductive material (e.g., a metal, an electrically conductive polymer, a semiconductor material doped sufficiently to become conductive, etc.). Grounding pad 144 may be disposed in or on flexible body 154. At least a first surface of grounding pad 144 may be exposed or external to flexible body 154 to enable the first surface to contact skin of a patient. Electrical connector 152 (e.g., a wire formed from electrically conductive material, etc.) may be coupled to grounding pad 144 and to a ground terminal 124. During performance of a high frequency ablation procedure, electrical charge in the patient's body may be extracted by grounding pad 144 and delivered to first ground terminal 124 via electrical connector 152, thereby preventing a potentially dangerous build-up of electrical charge in the patient and mitigating potentially harmful conditions from arising during the performance of a high frequency ablation procedure.

High frequency ablation device 102 may include one or more processors, such as real time processor 106, lagging processor 108, or both (referred to cumulatively as “processor 104”). Additionally, high frequency ablation device 102 may include memory 110, input/output devices 116, one or more sensors (referred to cumulatively as “sensors 118”), circuitry 120, and communication interface 122. Additionally, high frequency ablation device 102 may include first ground terminal 124, second ground terminal 126, one or more relays (cumulatively referred to as “relays 128”), and high frequency generator 130. Moreover, high frequency ablation device 102 may include temperature control device 132 and electrode 136. Electrode 136 may be placed within cannula 138, which is configured to be inserted within a patient's body for delivery of high frequency ablation therapy. Further, high frequency ablation device 102 may optionally include compressor 134. Although depicted as being incorporated into high frequency ablation device 102, first ground terminal 124 may be external to high frequency ablation device 102.

Processor 104 may include one or more microprocessors, graphical processing units (GPUs), field programmable gate arrays (FPGAs), microcontrollers, application specific integrated circuits (ASICs), and/or other logic circuitry. Processor 104 may be configured to execute an instruction set. In implementations, real time processor 106 may be configured to execute therapy related tasks, such as delivery of high frequency energy (e.g., RF energy) to a patient. Additionally, real time processor 106 may further be configured to perform safety related tasks, such as adjusting high frequency ablation therapy parameters of a high frequency ablation therapy. Moreover, real time processor 106 may be configured to execute system related tasks, such as monitoring an operational integrity of high frequency ablation device 102. Lagging processor 108 may be configured to execute input/output related tasks, such as responding to inputs received at input/output device 116, generating an output at input/output device 116, or both.

Memory 110 may include a random access memory (RAM), which can be synchronous RAM (SRAM), dynamic RAM (DRAM), synchronous dynamic RAM (SDRAM), or the like. Memory 110 may additionally or alternatively include read only memory (ROM), which can be programmable read only member (PROM), erasable programmable read only member (EPROM), electrically erasable programmable read only memory (EEPROM), optical storage, or the like. Additionally or alternatively, memory 110 may include hard disk drives (HDDs), solid state disk drives (SSDs), and other memory devices configured to store data, instructions, or both in a persistent or a non-persistent state. Memory 110 may be coupled to processor 104 and/or other components of high frequency ablation device 102. Memory 110 of embodiments may be a non-transitory computer-readable medium configured to store instructions, such as instructions 112, and/or data 114 utilized by processor 104 and/or other components of high frequency ablation device 102.

Instructions 112 may include software, firmware, or both. When executed by processor 104, instructions 112, or some portion thereof, may cause processor 104 to perform operations to control delivery of ablation therapy to a patient. These operations may include functions described more particularly with reference to FIGS. 1-5 as detailed below. As an example, instructions 112 may be configured to cause processor 104 to activate compressor 134 in response to receipt of data 114 indicating that grounding cuff 140 has been applied to a patient, or delivering RF energy to the patient according to parameters configured for the therapy, as described in more detail below.

Input/output devices 116 may include a keyboard, a mouse, a joystick, a touch-sensitive display, and/or other user interfaces, or any combination thereof. Additionally or alternatively, input/output device 116 may include a speaker configured to generate audible messages and/or alarms. Input/output device 116 may be configured to receive one or more inputs from a user of high frequency ablation device 102, to generate one or more outputs for the user of the high frequency ablation system 102, or both. As an example, input/output device 116 may be configured to render a graphical user interface (GUI) to receive inputs from and to render outputs for a user of high frequency ablation device 102 to facilitate configuration of parameters for high frequency ablation therapy, to provide outputs regarding a state of system 100, to provide outputs regarding a state of the patient, or any combination thereof. As an another example, input/output device 116, such as a speaker or a GUI, may be configured to generate alerts to cause a user of the system 100 to take one or more actions.

Sensors 118 may include one or more devices configured to measure one or more properties of the environment (e.g., temperature, pressure, voltage, current, etc.) and to convert the measured one or more properties to data 114 useable by processor 104. Sensors 118 may include electronics (e.g., analog electronics) configured to convert the measured one or more properties to an analog electrical signal. Additionally, in implementations, sensors 118 may further include electronics configured to convert the analog electrical signal to a digital electrical signal (e.g., analog to digital converters (ADCs)). Sensors 118 may be configured to detect, obtain, and/or provide data 114 to high frequency ablation device 102. For example, sensors 118 may include a thermocouple, a tachometer, a voltmeter, an ammeter, or any combination thereof. Although depicted as being illustrated in association with high frequency ablation device 102, sensor 118 also may be positioned within grounding pressure cuff 140, cannula 138, electrode, or a combination thereof. Sensors 118 may be coupled to processor 104 and/or other components of high frequency ablation device 102 via wired or wireless means. Sensors 118 may be configured to provide multiple data inputs to processor 104 in parallel. For example, a thermocouple, corresponding to sensor 118, may be configured to provide first input data (e.g., data 114) to processor 104 simultaneously with second input data (e.g., data 114) provided to processor 104 by a voltmeter.

Circuitry 120 may include various analog, digital, or mixed signal electronics configured to regulate an output voltage generated by high frequency generator 130. As configured to regulate an output voltage according to some examples, circuitry 120 may include a subtractor circuitry configured to calculate a temperature error as a difference between a target temperature and a measured temperature at a tip of cannula 138 (or electrode), a proportional-integral-derivative (PID) controller coupled to the subtractor circuit and configured to apply a high frequency voltage, such as an RF voltage, to the tip of cannula 138, the PID controller configured to determine the high frequency voltage based on the temperature error and a proportional coefficient, an integral coefficient, and a derivative coefficient of the PID controller. Circuitry 120 may further include a PID coefficient controller coupled to the PID controller, the PID coefficient controller configured to dynamically adjust the proportional, integral, and derivative coefficients of the PID controller during operation of high frequency ablation device 102. Circuitry 120 may be coupled to processor 104, to high frequency generator 130, or to both and/or to other components of high frequency ablation device 102.

Communication interface 122 may, for example, comprise a network interface card (NIC), a transceiver, a transmitter, a receiver, or any combination thereof. Additionally or alternatively, communication interface 122 may comprise networking hardware capable of communicating using the 802.11 communication standard, the Ethernet communication standard, other communication standards that may be developed, or any combination thereof. For example, communication interface 122 may be configured to receive and send data using a plurality of communication protocols, such as a Bluetooth™ protocol, a Zigbee™ protocol, a cellular communication protocol, such as any of the 3G, 4G, or 5G communication protocols, or any combination thereof. Communication interface 122 may be coupled to processor 104 and/or other components of high frequency ablation device 102. Communication interface 122 may be configured to receive data 114 transmitted wirelessly by sensor 118 included in or on grounding cuff 140.

As briefly described above, first ground terminal 124 may be configured to provide an electrical ground for grounding pad 144. For example, first ground terminal 124 may be a circuit configured to dissipate electric charge extracted from the patient by grounding pad 144. First ground terminal 124 may be formed from an electrically conductive material (e.g., a metal, an electrically conductive polymer, a semiconductor material sufficiently doped to become conductive, etc.) and may be coupled to grounding pad 144 of grounding cuff 140 via electrical connector 152. Although depicted as being a component of high frequency ablation device 102, first ground terminal 124 may be separate from (i.e., positioned outside of) high frequency ablation device 102. As shown in FIG. 1, second ground terminal 126 may be a circuit configured to provide an electrical ground for high frequency ablation device 102. Second ground terminal 126 may be formed from an electrically conductive material (e.g., a metal, an electrically conductive polymer, a semiconductor material sufficiently doped to become conductive, etc.).

High frequency generator 130 may be configured to generate and provide high frequency energy to the patient. For example, high frequency generator 130 may generate high frequency energy that may be delivered to a patient via electrode 136. In some implementations, high frequency generator 130 may be an RF generator. Relays 128 may include suitable components to controllably connect the output from high frequency generator 130 to one or more output channels. In some implementations, relays 128 may be implemented using mechanical switches to connect the RF signal from high frequency generator 130 to selected output channels. In some embodiments, relays 128 may be one or more switches such as, for example, one or more transistors, configured to selectively connect the output signal from high frequency generator 130 to one or more output channels for respective electrodes (e.g., electrode 136) such that multi-channel high frequency ablation therapy, such as RF ablation therapy, may be provided to a patient.

One or more electrodes, referred to as electrode 136, may be coupled to relays 128. Electrode 136 may be configured to deliver high frequency energy (e.g., RF energy) produced by high frequency generator 130 to neural tissue. In some aspects, the high frequency energy may be delivered to electrode 136 via relays 128. Cannula 138 may be used to position electrode 136 proximate to the target neural tissue. Electrode 136 further may be configured to be inserted within cannula 138. Cannula 138 may be a hollow needle configured to be inserted within the body of a patient, such as within a patient's spine. Electrode 136 may be threaded through the hollow space within cannula 138. Additionally, sensors 118 may be disposed within cannula 138. For example, a thermocouple may be disposed within cannula 138 to measure temperature proximate to neural tissue being ablated by RF energy delivered by electrode 138.

Temperature control device 132 may be configured to cool components of high frequency ablation device 102. Temperature control device 132 may, for example, be coupled to processor 104, to sensor 118, or to both and/or to other components of high frequency ablation device 102. As an example, temperature control device 132 may be a fan, a thermoelectric cooler, passive cooling material, etc.

In embodiments, high frequency ablation device 102 may optionally include compressor 134. Compressor 134 may be a motorized pump configured to pressurize a fluid (e.g., air) and may include an interface to connect with hose 150 to provide the pressurized fluid to inflatable bladder 142 via inlet 148. In lieu of compressor 134 or in addition to compressor 134, hand pump 160 may be used to deliver fluid to inflatable bladder 142 via hose 150 through inlet 148.

During operation of system 100, flexible body 154 of grounding cuff 140 may be fastened to a part of the patient's body (e.g., a patient's leg, arm, or torso) that is proximate to electrode 136 inserted within the patient's body. In some aspects, electrode 136 may be inserted within the patient's body via cannula 138. Once grounding cuff 140 is secured in position, inflatable bladder 142 may be inflated with a fluid (e.g. air) until a first surface of grounding pad 144 firmly contacts a patient's skin. Subsequently, high frequency ablation therapy may be delivered to the patient via high frequency ablation device 102 and electrode 136. After completion of the high frequency ablation therapy, valve 146 of inflatable bladder 142 may be opened to release the fluid (e.g., air) contained within the inflatable bladder 142, thereby relaxing grounding cuff 140. By maintaining firm contact between the first surface of grounding pad 144 and the patient's skin during delivery of the high frequency ablation procedure through pressure applied to grounding pad 144 by the fluid contained within inflatable bladder 142, an electrical connection can be sustained among the patient, the first surface of grounding pad 144, and first ground terminal 124. Moreover, the contact between the patient's skin and grounding pad 144 may not be impacted by sweat or the presence of hair, thereby overcoming several of the challenges associated with existing methods of providing grounding during ablation therapy.

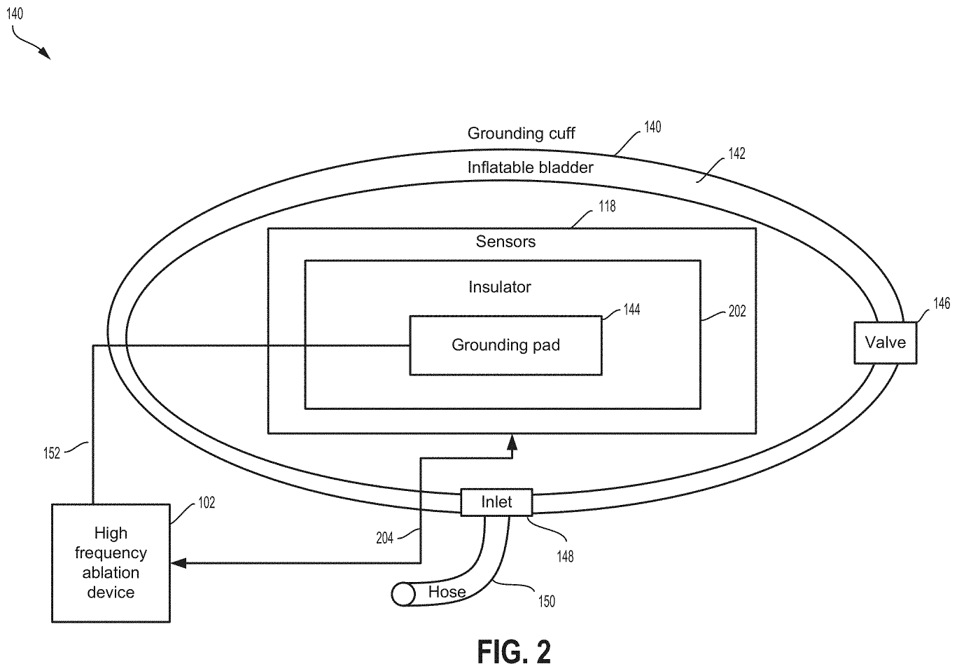

Sensors 118 (e.g., temperature sensors, piezoelectric sensors, pressure sensors, etc.) may be provided to monitor various metrics during a therapy session, such as a voltage at grounding pad 144, a temperature at a first surface of grounding pad 144 that is adjacent to the skin of the patient, an ambient temperature of grounding cuff 140, and a heart rate of the patient. It is noted that while FIG. 1 illustrates sensors 118 as being incorporated with or integrated into high frequency ablation device 102, in some implementations sensors 118 may be provided at grounding cuff 140. For example and referring to FIG. 2, a block diagram illustrating additional aspects of a grounding cuff according to embodiments of the present disclosure is presented. In addition to the components described with reference to FIG. 1, the grounding cuff of FIG. 2 includes sensors 118, insulator 202, and data bus 204.

Sensors 118 may include sensors configured to measure one or more states of grounding cuff 140 and its components, to measure one or more physiological characteristics of the patient, or both. For example, sensors 118 (e.g., one or more temperature sensors, piezoelectric sensors, pressure sensors, voltmeters, etc.) included in or on grounding cuff 140 may be configured to measure properties that relate to a state of grounding cuff 140, such as whether grounding cuff 140 has been attached to the patient, whether a first surface of grounding pad 144 contacts the patient's skin, an ambient temperature of the environment surrounding grounding cuff 140, whether electrical contact is maintained between grounding pad 144 and first ground terminal 124 of FIG. 1, a pressure at a second surface of grounding pad 144 that is adjacent to inflatable bladder 142, and the like. Additionally, sensors 118 (e.g., one or more heart rate sensors, temperature sensors etc.) included in or on grounding cuff 140 may be configured to measure one or more physiological characteristics of the patient, such as the patient's heart rate, blood pressure, body temperature, and the like.

Sensors 118 may be disposed in or around different components of grounding cuff 140, such as in or on flexible body 154, in or on inflatable bladder 142, or both. To elaborate, some of sensors 118 may be on an external surface of grounding cuff 140 (e.g., a surface facing away from the patient) while others of sensors 118 may be on an internal surface of grounding cuff 140 (e.g., a surface facing towards the patient). Examples of sensors that may be disposed on an internal surface of grounding cuff 140 may include a thermocouple configured to measure a temperature at the skin of the patient, a pressure sensor configured to measure a pressure exerted by inflatable bladder 142 on grounding pad 144, etc. Examples of sensors that may be disposed on an external surface of grounding cuff 140 may include a thermocouple configured to measure an ambient temperature of air surrounding grounding cuff 140. It is noted that the specific examples of sensors and sensor locations have been provided for purposes of illustration, rather than by way of limitation and that other types of sensors or sensor locations may be utilized by embodiments of the present disclosure.

Insulator 202 may be formed from a material configured to electrically isolate sensors 118 from grounding pad 144, which may prevent current extracted by grounding pad 144 from damaging one or more of sensors 118. For example, insulator 202 may be formed from a dielectric material (e.g., plastic, rubber, etc.) and may be positioned between sensor 118 and a second surface of grounding pad 144 (e.g., the second surface being opposite of the first surface of grounding pad 144).

Data bus 204 may provide a wired connection, a wireless connection, or both between sensors 118 and high frequency ablation device 102. Data bus 204 may be configured to send data 114 generated at sensors 118 to high frequency ablation device (e.g., via communication interface 122 of FIG. 1). In embodiments, high frequency ablation device 102 may also provide information, such as one or more control signals, to sensors 118 via data bus 204. Moreover, in embodiments, data bus 204 may be a wired connection that is bundled with electrical connector 152 of FIG. 2.

During operation of system 100 of FIG. 1 with grounding cuff 140 of FIG. 2, sensors 118 may provide data 114 to processor 104 of high frequency ablation device 102 via communication interface 122. Based on receipt of data 114, processor 104 may be configured to generate control signals. The control signals may be configured to effectuate one or more actions to facilitate high frequency ablation therapy. For example, a first sensor of sensors 118 (e.g., a thermocouple) embedded in grounding cuff 140 may record a temperature (e.g., the patient's body temperature between approximately 36.1° C. to 37.2° C.) indicating that grounding cuff 140 has been attached to the patient. The first sensor may send data 114 (e.g., wirelessly, through wired means, or both) to processor 104 via communication interface 122. Instructions 112 may cause processor 104 to analyze data 114, and processor 104 may determine, based on the analysis, that grounding cuff 140 has been attached to the patient. In response to the determination, instructions 112 may cause processor 104 to send a first control signal to cause compressor 134 to activate, thereby filling inflatable bladder 142 with a volume of air. A second sensor of sensors 118 embedded in grounding pad 144 (e.g., a piezoelectric sensor, a voltmeter, etc.) may measure a pressure at grounding pad 144, a voltage at grounding pad 144, or both, indicating that the first surface of grounding pad 144 contacts the skin of the patient and that grounding pad 144 is electrically coupled to a first ground terminal (e.g., first ground terminal 124). In response to receipt of data 114, sent by the second sensor to processor 104 via communication interface 122, processor 104 may be configured to send a second control signal to cause high frequency generator 130 to provide high frequency energy (e.g., RF energy) to electrode 136.

In response to the second control signal, high frequency ablation device 102 may deliver high frequency ablation therapy to a patient. For example, high frequency generator 130 may be configured to provide high frequency energy (e.g., RF energy) to one or more electrodes (e.g., electrode 136) coupled to high frequency generator 130. In some aspects, one or more relays (e.g., relays 128) may be utilized to deliver the high frequency energy to the one or more electrodes. Moreover, sensors 118 disposed in or on grounding cuff 140 may be configured to monitor a state of grounding pad 144 during delivery of high frequency ablation therapy. For example, sensors 118 disposed in or on grounding cuff 140, such as an impedance sensor, a piezoelectric sensor, etc., may collect data 114 indicative of a condition in which grounding pad 144 fails to make sufficient electrical contact with the skin of the patient. In response to receipt of such data 114, processor 104 may cause high frequency generator 130 to cease providing radio frequency (RF) energy to electrode 136, to send an alert to input/output device 116 notifying a clinician that grounding pad 144 has become unmoored, or both. In some implementations, in response to receipt of data 114 indicating that grounding pad 144 has ceased to make adequate contact with the skin of the patient, processor 104 may cause high frequency ablation device 102 to cease providing high frequency ablation therapy until a clinician reconfigures grounding cuff 140 so that the first surface of grounding pad 144 adequately contacts the skin of the patient.

As another example and during delivery of high frequency ablation therapy, sensors 118 (e.g., a heart rate sensor etc.) in or on grounding cuff 140 may measure a heart rate of the patient. In response to receipt of data 114, corresponding to the patient's heart rate, processor 104 may be configured to generate a third control signal configured to dynamically adapt parameters of the high frequency ablation therapy, such as an output voltage generated by high frequency generator 130. By dynamically adapting parameters of the high frequency ablation therapy in response to real time data received from sensors 118 in or on grounding cuff 140, a quality of the high frequency ablation therapy may be enhanced. Alternatively or additionally, in response to receipt of data 114 corresponding to the patient's heart rate, processor 104 may be configured to generate a control signal configured to stop delivery of the high frequency ablation therapy. It is noted that using a sensor to measure a heart rate and adjusting control parameters for high frequency ablation based on the heart rate has been described for purposes of illustration, rather than by way of limitation and that other types of physiological parameters and sensors may be utilized in accordance with the present disclosure.

In some embodiments, upon completion of the high frequency ablation therapy, processor 104 may be configured to send a control signal to cause valve 146 of inflatable bladder 142 to open. The opening of valve 146 may cause release of fluid (e.g., air) contained within the inflatable bladder 142, thereby causing inflatable bladder 142 to deflate. Alternatively or additionally, processor 104 may cause input/output device 116 to display a human-readable message or audible alert indicating to a user of high frequency ablation device 102 that valve 146 should be opened to release the fluid (e.g., air) from inflatable bladder 142.

System 100 confers numerous advantages. As an example, system 100 may enhance a safety of high frequency ablation procedures performed using high frequency device 102. By securely contacting a first surface of grounding pad 144 to a patient's skin throughout the duration of a high frequency ablation (e.g., an RF ablation) procedure, the possibility of discomfort, severe injury, or both is reduced. Additionally, in implementations, by monitoring one or more physiological parameters of the patient, one or more physical properties of grounding pad 144, or both, sensors 118 in or on grounding cuff 140 may provide data 114 to high frequency ablation device 102 to effectuate dynamic and automatic adjustment of high frequency ablation therapy.

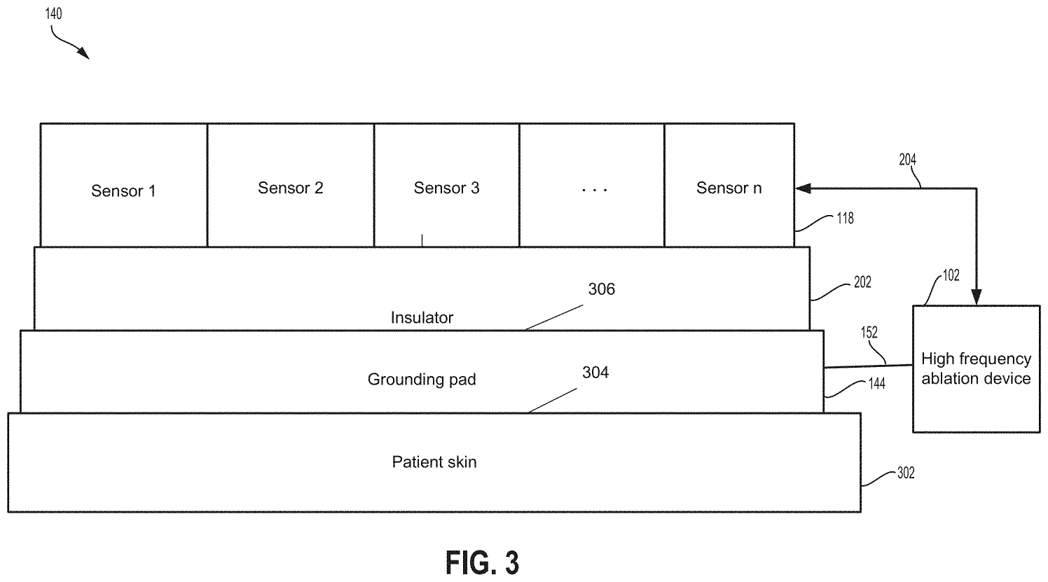

Referring to FIG. 3, a block diagram illustrating additional aspects of a grounding cuff 140 according to embodiments of the present disclosure is shown. Grounding cuff 140 may include grounding pad 144, insulator 202, and sensor 118. First surface 304 of grounding pad 144 may be configured to contact a surface of patient skin 302. Second surface 306 of grounding pad may be disposed adjacent to or coupled to insulator 202. Insulator 202 may be disposed adjacent to or coupled to sensors 118. As depicted in FIG. 3, sensors 118 may include a plurality of sensors, designated as sensors 1−n, where n≥1. As described above, the n sensors may include sensors configured to measure properties or an operational status of grounding cuff 140, measure physiological properties of the patient, or both.

In a non-limiting example, the n sensors may include a first set of sensors configured to measure a characteristic (e.g., a voltage, an impedance, etc.) of grounding pad 144 to determine that grounding pad is electrically coupled to a first ground terminal (e.g., first ground terminal 124 of FIG. 1), a second sensor configured to measure a temperature of the grounding pad to detect, for example, improper heating at grounding pad 144, and a third sensor configured to measure an ambient temperature of grounding cuff 140. Moreover, the n sensors may include a piezoelectric sensor to detect a pressure exerted on grounding pad 144 by the inflatable bladder (not depicted in FIG. 3). It is noted that the non-limiting examples of sensors that may be used with a grounding cuff described above have been provided by way of illustration, rather than by way of limitation and that other types of sensors or combinations of sensor may be utilized with grounding cuffs in accordance with the concepts disclosed herein.

As described above, the plurality of sensors (e.g., sensors 118) may be configured to send data (e.g., data 114 of FIG. 1) to high frequency ablation device 102 via data bus 204. Additionally or alternatively, the plurality of sensors may be configured to send data wirelessly to high frequency ablation device 102 using a Bluetooth™ protocol; a Zigbee™ protocol; a cellular communication protocol, such as any of the 3G, 4G, and/or 5G communication protocols; and/or a 802.11 communication standard. As an example and referring to FIG. 1, a first sensor of the first plurality of sensors 118 may send data 114 corresponding to a temperature at grounding pad 144 to communication interface 122 of high frequency ablation device 102. A second sensor of the first plurality of sensors 118 may send data 114 corresponding to an ambient temperature of grounding cuff 140 to communication interface 122 of high frequency ablation device 102. Instructions 112 may cause processor 104 to determine a difference between the temperature at grounding pad 144 and the ambient temperature of grounding cuff 140. If the difference exceeds a threshold value (e.g., stored in memory 110), processor 104 may cause input/output device 116 to render a human-readable message, audible alarm alert, or both notifying the user that grounding cuff 140 might be malfunctioning. Additionally or alternatively, processor 104 may cease providing energy to the electrode 136 upon detecting that the difference exceeds the threshold value.

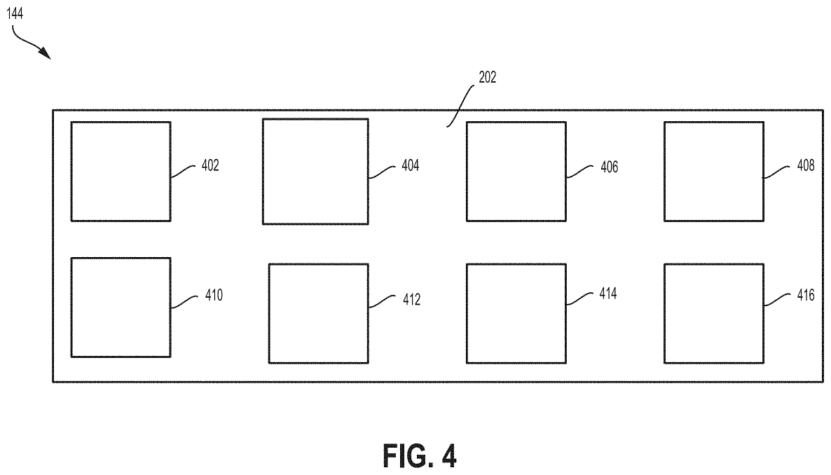

Referring to FIG. 4, a block diagram illustrating aspects of grounding pad 144 according to embodiments of the present disclosure is shown. Grounding pad 144 may be a component of a grounding cuff, such as grounding cuff 140 of FIGS. 1-3. Grounding pad 144 may include a plurality of grounding pad sections 402-416. Each grounding pad section 402-416 may be physically separated and electrically isolated from every other grounding pad section 402-416. For example, a grounding pad section may be separated from other grounding pad sections by a threshold distance. In an aspect, the threshold distance may be less than 1 millimeters (mm), 1-5 mm, 1-10 mm, 10-25 mm, 10-100 mm, or another distance. A second surface of each grounding pad section 402-416 may be configured to contact insulator 202 and a first surface of each grounding pad section 402-416 may be configured to contact the skin of the patient. Each grounding pad section 402-416 may be coupled to an electrical connector (not depicted) configured to couple each grounding pad section 402-416 to a first ground terminal (e.g., first ground terminal 124 of FIG. 1). In some aspects, at least one sensor (e.g., sensors 118 not depicted) may be provided in association with each grounding pad section 402-416, as described above. Where sensors are provided, the grounding cuff may include a data bus (not depicted) configured to provide data (e.g., data 114 of FIG. 1) generated by the at least one sensor (e.g., sensors 118 of FIGS. 1-3) to one or processors (e.g., processor 104 of FIG. 1) of a high frequency ablation device (e.g., high frequency ablation device 102) via one or more communication interfaces (e.g., communication interface 122 of FIG. 1).

During typical operation, each first surface of each grounding cuff section 402-416 may contact the skin of the patient due to pressure exerted by a volume of fluid (e.g., air) contained within the inflatable bladder (e.g., inflatable bladder 142 of FIGS. 1-2). Since each grounding cuff section 402-416 is physically distinct from every other grounding cuff section 402-416, each grounding cuff section 402-416 may contact a different portion of the skin of the patient from every other grounding cuff section 402-416. By dividing grounding pad 144 into an array having a plurality of grounding pad sections 402-416, data may be obtained about conditions at each segment of a patient's skin that contacts the respective grounding pad section (e.g., ground pad sections 402-416). Such data may provide a more accurate and precise understanding of a state of grounding pad 144 itself and of a physiological condition of the patient than a unitary grounding pad (i.e., a grounding pad without grounding pad sections). Although grounding cuff sections 402-416 are depicted as being squares, grounding cuff sections 402-416 may have any geometry (e.g., circular geometry, rectangular geometry, triangle geometry, other geometries, or combinations thereof). In some aspects, one or more if grounding cuff sections 402-416 may have a different geometry from other ones of grounding cuff sections 402-416. Further, although grounding cuff sections 402-416 are depicted as having similar dimensions, in implementations, grounding cuff sections 402-416 may have non-uniform dimensions with respect to each other.

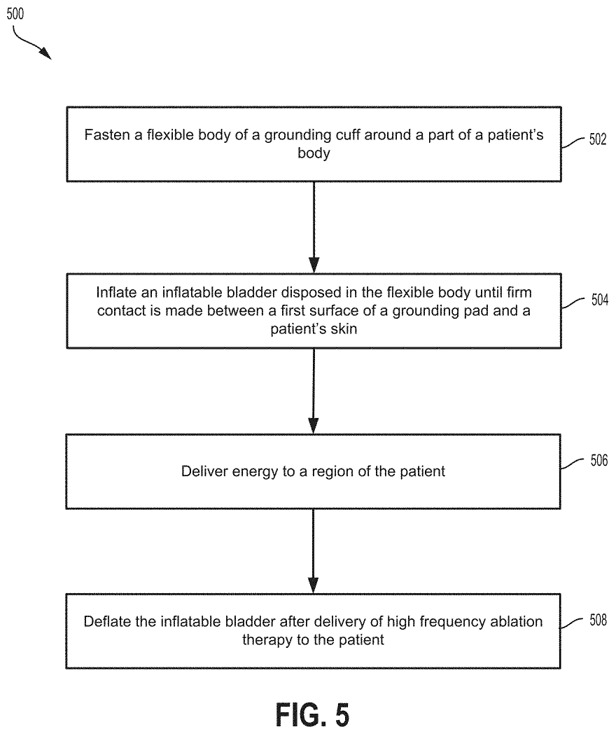

Referring to FIG. 5, a flow diagram of an exemplary method for using a grounding cuff according to embodiments of the present disclosure is shown as method 500. At block 502, method 500 includes fastening a flexible body of a grounding cuff around a part of a patient's body. At block 504, method 500 includes inflating an inflatable bladder disposed in the flexible body to a threshold inflation level. As described above, the threshold inflation level may correspond to a level of inflation configured to promote firm contact between a first surface of a grounding pad of the grounding cuff and the patient's skin. At block 506, method 500 includes delivering energy to a region of the patient. As described above, the energy may be delivered to the patient via an electrode inserted into the patient's body. At block 508, method 500 includes deflating the inflatable bladder (e.g., after delivery of high frequency ablation therapy to the patient).

To elaborate, at block 502, one end of the flexible body of the grounding cuff (e.g., first end 162, second end 164 of FIG. 1) may be attached to the other end of the flexible body of the grounding cuff (e.g., first end 162, second end 164 of FIG. 1). A fastener, such as fastener 158 of FIG. 1, may be used to hold the one end of the flexible body to the other end of the flexible body. In this manner, the grounding cuff maybe firmly but comfortably fastened around a part of a patient's body that is proximate to a site of the patient's body at which one or more cannulas containing electrodes are placed. For example, the flexible body may be wrapped around and attached to a patient's leg, arm, or torso.

At block 504, the inflatable bladder disposed in the flexible body may be automatically inflated until firm contact is made between a first surface of a grounding pad of the grounding cuff and the patient's skin. In an embodiment, one or more sensors disposed in or on the grounding cuff may send data to a communication interface of a high frequency ablation device (e.g., a radio frequency (RF) ablation device) indicating that the grounding cuff has been fastened to the patient. For example, a sensor of the one or more sensors may be a temperature sensor configured to monitor a temperature near a first surface of the grounding pad of the grounding cuff. When the grounding cuff has been fastened to the patient, a temperature reading proximate to the first surface of the grounding pad may be between 36.1° C. and 37.2° C. (i.e., human body temperature), indicating that the grounding cuff has been affixed to the patient. In response to receipt, at one or more processors of the high frequency ablation device, of the temperature data, instructions, stored in a memory of the high frequency ablation device, may cause the one or more processors to send a control signal to a compressor attached to the inflatable bladder of the grounding cuff. In response to receipt of the control signal, the compressor may fill the inflatable bladder with air. Alternatively or additionally, the one or more processors may be configured to generate an alert on an input/output device of the high frequency ablation device indicating to the user of the high frequency ablation device that the grounding cuff is properly positioned and that the inflatable bladder should be inflated.

The grounding cuff may further include sensors to detect that a substantial surface area of the first surface of the grounding pad firmly contacts the skin of the patient. The sensors may send the data to the communication interface of the high frequency ablation device (e.g., through a data bus, wirelessly, or both). The one or more processors of the high frequency ablation device may use the data to calculate a grounding metric corresponding to the quality of contact between the first surface of the grounding pad and the patient's skin. The one or more processors may further be configured to compare a value of the grounding metric to a threshold value stored, for instance, in a memory of the high frequency ablation device. In response to the value of the grounding metric at least equaling or exceeding the threshold value, the one or more processors may send a control signal to the compressor to instruct the compressor to stop pumping pressurized air into the inflation bladder.

Throughout delivery of high frequency ablation therapy to the patient, at block 508, the sensors may collect data relating to the quality of contact between the grounding pad and the patient's skin, and the one or more processors may be configured to dynamically evaluate the data so that grounding metric can be dynamically calculated during delivery of high frequency ablation therapy. In response to detecting a material change in the value of the grounding metric, the one or more processors may cause an input/output device of the high frequency ablation device to generate an alert (e.g., an audible alert, a visual alert, or both) to warn a user of the high frequency ablation device that the quality of the contact between the first surface of the grounding pad and the patient's skin has deteriorated. Additionally or alternatively, if a difference between the value of the grounding metric and the threshold value is within a designated range (e.g., indicating a slight deterioration in the quality of the contact between the first surface of the grounding pad and the patient's skin), the one or more processors may be configured to cause the compressor to re-inflate the inflatable bladder to dynamically adjust pressure applied to the first surface of the grounding pad. In embodiments, the sensors to detect that a substantial surface area of the first surface of the grounding pad firmly contact the patient's skin may correspond to a pressure sensor (e.g., a piezoelectric sensor) configured to measure a pressure at the first surface of the grounding pad, a voltmeter configured to measure a first voltage at the first surface of the grounding pad and a second voltage at a first ground terminal of the high frequency ablation device, an impedance sensor configured to measure at impedance at the first surface of the grounding pad, or any of the foregoing.

At block 508, the inflatable bladder disposed in the flexible body may be deflated after delivery of high frequency ablation therapy to a patient. For example, in response to detecting that a high frequency ablation therapy session is complete, the one or more processors of the high frequency ablation device may cause a valve of the inflatable bladder to open, thereby releasing a volume of air stored in the bladder. Alternatively or additionally, a user may manually deflate the inflatable bladder via a valve, as described above.

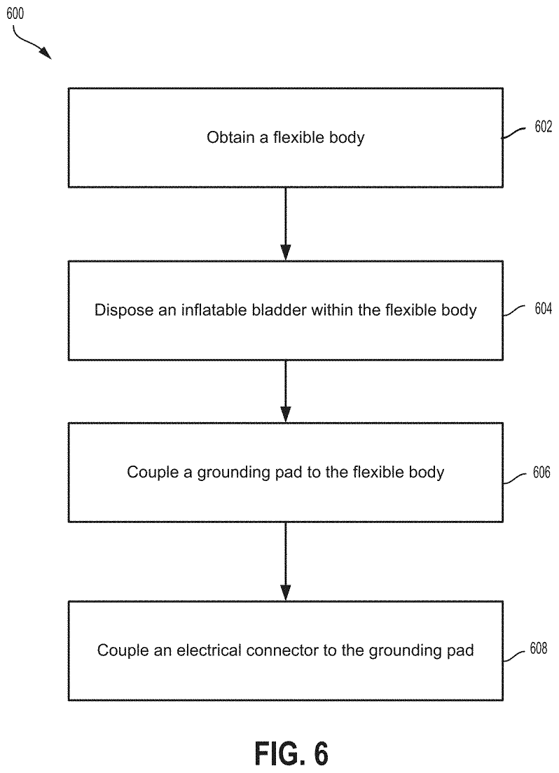

FIG. 6 is a flow diagram of a method 600 for manufacturing a ground cuff according to embodiments of the present disclosure. At block 602, a flexible body is obtained. At block 604, an inflatable bladder is disposed within the flexible body. At block 606, a grounding pad is coupled to the flexible body. At block 608, an electrical connector is coupled to the grounding pad.

To elaborate, at block 602, a flexible body (e.g., flexible body 154 of FIG. 1) may be obtained having characteristics suitable for wrapping around a portion of the patient's body that is proximate to the site at the patient's body at which high frequency ablation therapy is to be delivered. The flexible body may be fabricated from a durable material, such as cotton, nylon, polyester, etc. The flexible body may have a first end and a second end configured to attach to one another. A fastener (e.g., fastener 158 of FIG. 1) may be positioned at one of the ends or at both ends and configured to hold the first end to the second end so that that the flexible body can be held securely and tightly to a portion of the patient's body that is proximate to the site of the patient's body at which high frequency ablation therapy is to be delivered.

At block 604, an inflatable bladder (e.g., inflatable bladder 142 of FIG. 1) may be disposed within the flexible body. For example, the inflatable bladder may inserted within an opening of the flexible body, which after insertion of the inflatable bladder may be closed (e.g., using mechanical means such as stitches). Additionally or alternatively, the inflatable bladder may be attached to a surface of the flexible body by adhesive or mechanical means (e.g., screws, staples, stiches, etc.). In implementations, one or more sensors may be embedded in or on the flexible body, in or on a surface of the inflatable bladder, or any combination thereof.

At block 606, a grounding pad (e.g., grounding pad 144 of FIG. 1) may be coupled to the flexible body. For example, the grounding pad may be attached to an electrically insulating surface separating the grounding pad from the one or more sensors. For instance, the grounding pad may be attached to the electrically insulating surface by adhesive or mechanical means (e.g., screws, staples, stiches, etc.). In this manner, the electrically insulating surface may be positioned between the one or more sensors and the grounding pad.

At block 608, an electrical connector (e.g., electrical connector 152 of FIG. 1) may be coupled to the grounding pad. The electrical connector may have a first end configured to be attached to the grounding pad. A second end of the electrical connector may be configured to attach to a ground terminal (e.g., first ground terminal 124 of FIG. 1). For example, a first end of the electrical connector may be soldered onto the grounding pad, and a second end of the electrical connector may have a connector (e.g., a UBS connector, etc.) capable of attaching to a first ground terminal.

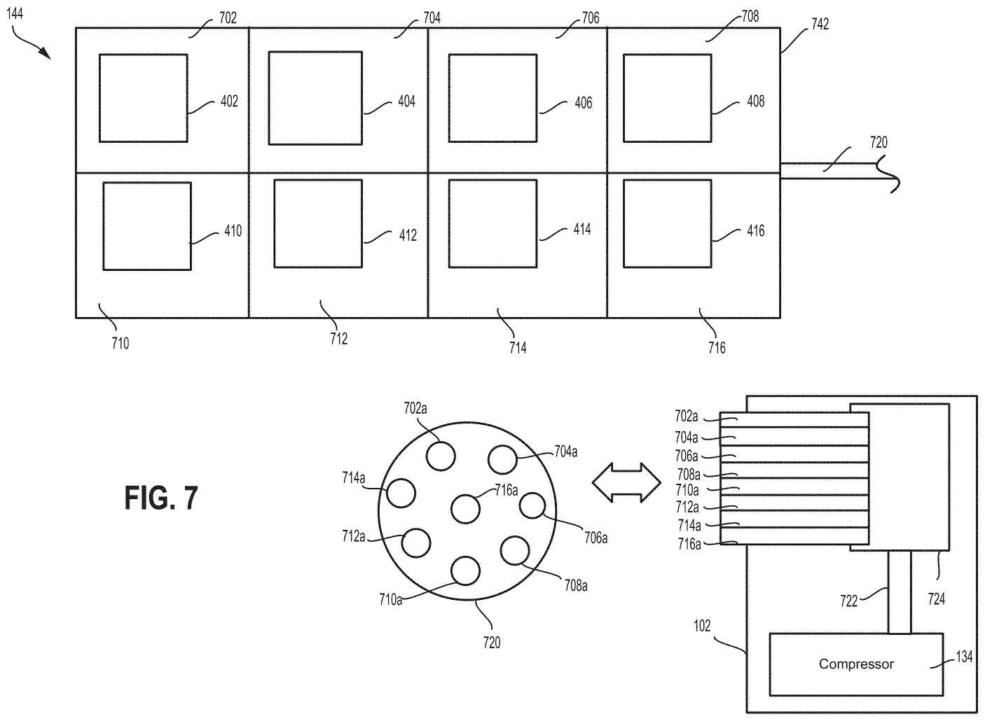

Referring to FIG. 7, a block diagram illustrating additional aspects of a grounding cuff according to embodiments of the present disclosure is shown. In the exemplary grounding cuff of FIG. 7, the sectional grounding pad 144 of FIG. 4 is shown. As described above, the sectional grounding pad 144 may include a plurality of electrically isolated grounding pad sections 402-418. However, unlike the grounding cuff of FIG. 4, in the exemplary embodiment of FIG. 7 also includes a sectional inflatable bladder 742. The sectional inflatable bladder 742 may include a plurality of inflatable bladder sections 702-716, each of which may be associated with a corresponding one of the grounding pad sections 402-418. For example, grounding pad section 402 corresponds to inflatable bladder section 702, grounding pad section 404 corresponds to inflatable bladder section 704, grounding pad section 406 corresponds to inflatable bladder section 706, grounding pad section 408 corresponds to inflatable bladder section 708, grounding pad section 410 corresponds to inflatable bladder section 710, grounding pad section 412 corresponds to inflatable bladder section 712, grounding pad section 414 corresponds to inflatable bladder section 714, and grounding pad section 416 corresponds to inflatable bladder section 716. Each inflatable bladder section 702-716 may be inflated with a particular volume of fluid that might be the same as or different from a volume of fluid to which one or more other inflatable bladder sections 702-716 is inflated. In this manner, particularized control over an extent of contact between a first surface of each grounding pad section 402-416 and a corresponding segment or region of skin of a patient may be effectuated, since a quantity of pressure exerted on the corresponding segment or region of the skin of the patient by each inflatable bladder section 702-716 may be uniquely controlled.

To implement the foregoing particularized control, each inflatable bladder section 702-716 may be associated with a particular hose of hoses 702a-716a. For instance, each inflatable bladder section 702-716 may include an inlet (not depicted) to which one of hoses 702a-716a may attach. Further, each inflatable bladder section 702-716 may include a valve (not depicted) to permit controlled ingress and/or egress of fluid from a corresponding one of hoses 702a-716a to a corresponding inflatable bladder section 702-716. Moreover, hoses 702a-716a may be contained within hose 720 with each hose of hoses 702a-716a separately and independently leading to a particular inflatable bladder section 702-716. Further, each hose of hoses 702a-716b may be configured to attach to hose switch and pressure monitoring device 724. Hose switch and pressure monitoring device 724 may be configured to attach to fluid chamber 722. Fluid chamber 722 may be configured to attach to compressor 134. In an implementation, compressor 134, fluid chamber 722, and hose switch and pressure monitoring device 724 may be components of a high frequency ablation device (e.g., high frequency ablation device 102 of FIG. 1). However, in other implementations, compressor 134, fluid chamber 722, and hose switch and pressure monitoring device 724 may be separate from high frequency ablation device 102. In some aspects, fluid chamber 722 may be omitted and fluid may be pumped into the hoses 702a-716a directly by compressor 134 and hose switch and pressure monitoring device 724.

During operation, logic of hose switch and pressure monitoring device 724 (e.g., software, firmware, or both contained within hose switch and pressure monitoring device 724), processor 104 of FIG. 1 of high frequency ablation device 102 implementing instructions 112 of FIG. 1, or both may be configured to cause compressor 134 to provide one or more hoses 702a-716a with particular quantities of fluid (e.g., air). One or more hoses 702a-716 likewise may provide the fluid (e.g., air) to one or more corresponding inflatable bladder sections 702-716, thereby causing the one or more bladder sections 702-716 to inflate and apply a particularized pressure to corresponding ground pad sections 402-416. One or more sensors (e.g., sensors 118 of FIGS. 1-3), such as one or more pressure sensors, corresponding to the one or more ground pad sections 402-416 may relay data (e.g., pressure data) associated with the one or more ground pad sections 402-416 to logic of hose switch and pressure monitoring device 724, processor 104 of FIG. 1 of high frequency ablation device 102, or both, as described above. Based on the data provided by the one or more pressure sensors, logic of hose switch and pressure monitoring device 724, processor 104 of FIG. 1 of high frequency ablation device 102, or both may be configured to initiate operation of compressor 134 to individually adjust a quantity of fluid contained within each inflatable bladder sections 702-716.

For example, in response to receipt of data from one more sensors indicating sub-optimal contact between ground pad section 404 and ground pad section 414 and the corresponding portions of the skin of the patient, logic of hose switch and pressure monitoring device 724, processor 104 of FIG. 1 of high frequency ablation device of FIG. 1, or both may be configured to initiate operation of compressor 134 to provide fluid (e.g., air) through hoses 704a and 714a to inflatable bladder sections 704 and 714. A first amount of fluid provided to inflatable bladder section 704 may be different from a second amount of fluid provided to inflatable bladder section 714, since a first amount of pressure to enhance contact between ground pad section 404 and the corresponding segment of the patient's skin may be different from a second amount of pressure to enhance contact between ground pad section 414 and the corresponding segment of the patient's skin. By dynamically tuning a quantity of fluid provided to each inflatable fluid bladder section 702-716 in response to data received from one or more sensors (e.g., sensors 118 of FIGS. 1-3), enhanced contact can be established between a first surface of each ground pad section 402-416 and the corresponding segment of the patient's skin thereby enhancing delivery of high frequency ablation therapy to the patient. Additionally, the ability to provide individualized control over the contact between each grounding pad section 702-716 and the skin of the patient may enable the grounding cuff shown in FIG. 7 to provide optimal contact over irregular body contours, which may enable the ground cuff to be disposed at locations on a patient that were not feasible using previous grounding technologies that relied on adhesives and grounding pads having a singular body construction.

It is noted that using data received from pressure sensors to customize the quantity of fluid delivered to each inflatable bladder section 702-716 has been described for purposes of illustration, rather than by way of limitation and that other types of sensors may be utilized, individually or in combination with pressure sensors, to customize the quantity of fluid delivered to each inflatable bladder section. For example, sensor data associated with impedance measurements captured for each grounding pad section 402-416 may be utilized to evaluate the level of contact between each individual grounding pad section 402-416 and the skin of the patient, and adjustments may be made to increase the amount of fluid delivered to inflatable bladder sections corresponding to grounding pad sections estimated to have insufficient contact with the skin of the patient based on the impedance measurements. Additionally, the quantity of fluid delivered to each inflatable bladder section 702-716 may also be controlled manually, rather than under automated control of logic of hose switch and pressure monitoring device 724, processor 104 of FIG. 1 of high frequency ablation device of FIG. 1, or both, such as via switches or controls provided to a user to manually control compressor 134 and hose switch and pressure monitoring device 724 to deliver fluid to specific inflatable bladder sections until a desired level of inflation is achieved.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Components, the functional blocks, and the modules described herein with respect to FIGS. 1-7) include processors, electronics devices, hardware devices, electronics components, logical circuits, memories, software codes, firmware codes, among other examples, or any combination thereof. In addition, features discussed herein may be implemented via specialized processor circuitry, via executable instructions, or combinations thereof.

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Skilled artisans will also readily recognize that the order or combination of components, methods, or interactions that are described herein are merely examples and that the components, methods, or interactions of the various aspects of the present disclosure may be combined or performed in ways other than those illustrated and described herein.

The various illustrative logics, logical blocks, modules, circuits, and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules, and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or any conventional processor, controller, microcontroller, or state machine. In some implementations, a processor may also be implemented as a combination of computing devices, such as a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or any combination thereof. Implementations of the subject matter described in this specification also may be implemented as one or more computer programs, that is one or more modules of computer program instructions, encoded on a computer storage media for execution by, or to control the operation of, data processing apparatus.

If implemented in software, the functions may be stored on or transmitted over as one or more instructions or code on a computer-readable medium. The processes of a method or algorithm disclosed herein may be implemented in a processor-executable software module which may reside on a computer-readable medium. Computer-readable media includes both computer storage media and communication media including any medium that may be enabled to transfer a computer program from one place to another. A storage media may be any available media that may be accessed by a computer. By way of example, and not limitation, such computer-readable media can include random-access memory (RAM), read-only memory (ROM), electrically erasable programmable read-only memory (EEPROM), CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, hard disk, solid state disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to some other implementations without departing from the spirit or scope of this disclosure. Moreover, while specific embodiments have been illustrated with respect to FIGS. 1-7 to highlight specific features of grounding cuffs according to the present disclosure, it should be understood that features and concepts described with respect to one of the different illustrated embodiments may be combined or used with other embodiments. Thus, the claims are not intended to be limited to the implementations shown herein, but are to be accorded the widest scope consistent with this disclosure, the principles and the novel features disclosed herein.

Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of any device as implemented.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted may be incorporated in the example processes that are schematically illustrated. For example, one or more additional operations may be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, some other implementations are within the scope of the following claims. In some cases, the actions recited in the claims may be performed in a different order and still achieve desirable results.

As used herein, including in the claims, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. The term “or,” when used in a list of two or more items, means that any one of the listed items may be employed by itself, or any combination of two or more of the listed items may be employed. For example, if a composition is described as containing components A, B, or C, the composition may contain A alone; B alone; C alone; A and B in combination; A and C in combination; B and C in combination; or A, B, and C in combination. Also, as used herein, including in the claims, “or” as used in a list of items prefaced by “at least one of” indicates a disjunctive list such that, for example, a list of “at least one of A, B, or C” means A or B or C or AB or AC or BC or ABC (that is A and B and C) or any of these in any combination thereof. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed aspect, the term “substantially” may be substituted with “within [a percentage] of” what is specified, where the percentage includes 0.1, 1, 5, and 10 percent; and the term “approximately” may be substituted with “within 10 percent of” what is specified. The phrase “and/or” means and or.

Although the aspects of the present disclosure and their advantages have been described in detail, it should be understood that various changes, substitutions and alterations can be made herein without departing from the spirit of the disclosure as defined by the appended claims. Moreover, the scope of the present application is not intended to be limited to the particular implementations of the process, machine, manufacture, composition of matter, means, methods and processes described in the specification. As one of ordinary skill in the art will readily appreciate from the present disclosure, processes, machines, manufacture, compositions of matter, means, methods, or operations, presently existing or later to be developed that perform substantially the same function or achieve substantially the same result as the corresponding aspects described herein may be utilized according to the present disclosure. Accordingly, the appended claims are intended to include within their scope such processes, machines, manufacture, compositions of matter, means, methods, or operations.

What is claimed is:

1. A grounding cuff comprising:
a flexible body;
an inflatable bladder disposed within the flexible body, wherein the inflatable bladder is configurable to an inflated state and a deflated state, wherein the inflatable bladder comprises an inlet configured to receive a fluid from a hose, and wherein the inflatable bladder is configured to the inflated state based on the fluid received via the hose;
a grounding pad coupled to the inflatable bladder and coupled to a high frequency ablation device, wherein at least a first surface of the grounding pad comprises an electrically conductive material and is configured to contact skin of a patient; and
an electrical connector configured to couple the grounding pad to a first ground terminal.

2. The grounding cuff of claim 1, wherein the inflatable bladder includes a valve configured to release the fluid from the inflatable bladder to configure the inflatable bladder to the deflated state, and wherein the grounding cuff further comprises:
one or more sensors; and
an insulating layer positioned between the one or more sensors and a second surface of the grounding pad, wherein the one or more sensors are positioned between the insulating layer and the inflatable bladder, wherein the insulating layer is configured to provide electrical insulation between the grounding pad and the one or more sensors.

3. The grounding cuff of claim 2, further comprising a data bus configured to transmit data collected from the one or more sensors to one or more processors of the high frequency ablation device, and wherein the one or more sensors comprise at least a first sensor configured to measure a quality of a contact between the first surface of the grounding pad and the skin of the patient.

4. The grounding cuff of claim 3 wherein the one or more sensors further comprise at least a second sensor configured to monitor a physiological state of the patient while high frequency ablation therapy is administered to the patient via the high frequency ablation device, and wherein the at least the second sensor is further configured to transmit data corresponding to the physiological state of the patient to the high frequency ablation device via a data bus configured to couple the one or more sensors to a communication interface of the high frequency ablation device.

5. The grounding cuff of claim 3, wherein the one or more sensors comprise a first temperature sensor configured to monitor a temperature of a region at which the ground pad contacts the skin of the patient and a second temperature sensor configured to monitor a temperature of ambient air surrounding the patient, a pressure sensor configured to monitor a pressure of fluid within the inflation bladder, a heart-rate sensor configured to monitor a heart-rate of the patient, and a blood pressure sensor configured to monitor a blood pressure of the patient.

6. The grounding cuff of claim 2, wherein the grounding pad comprises a plurality of grounding pad sections, wherein each grounding pad section of the plurality of grounding pad sections is electrically isolated from every other grounding pad section, wherein the one or more sensors include at least one sensor corresponding to each of the one or more grounding pad sections, and wherein the first surface is comprised of a plurality of surfaces, each surface of the plurality of surfaces configured to contact a different portion of the skin of the patient.

7. The grounding cuff of claim 6, wherein the one or more sensors are configured to measure an extent to which each grounding pad section contacts the different portion of the skin of the patient.

8. The grounding cuff of claim 1, wherein coupling between the grounding pad and the skin of the patient is configured to prevent a buildup of electrical charge in a patient while high frequency ablation therapy is administered to the patient.

9. The grounding cuff of claim 1, wherein the first ground terminal is electrically isolated from a second ground terminal providing grounding to the high frequency ablation device.

10. A method of manufacturing a grounding cuff, the method comprising:
obtaining a flexible body;
disposing an inflatable bladder within the flexible body, wherein the inflatable bladder comprises an inlet configured to receive a fluid, and wherein the inflatable bladder is configurable to an inflated state and a deflated state;
coupling a grounding pad to the flexible body, wherein the grounding pad comprises an electrically conductive material having at least a first surface that is disposed external to the flexible body and configured to contact skin of a patient; and
coupling an electrical connector to the grounding pad, wherein the electrical connector is configured to couple the grounding pad to a first ground terminal.

11. The method of claim 10, further comprising:
positioning an insulating layer between one or more sensors and a second surface of the grounding pad; and
disposing the one or more sensors between the surface of the inflatable bladder and the insulating layer, wherein the insulating layer is configured to electrically insulate the one or more sensors and the grounding pad.

12. The method of claim 11, further comprising communicatively coupling the one or more sensors to one or more processors of a high frequency ablation device, wherein the coupling is effectuated wirelessly or through a data bus.

13. The method of claim 11, wherein the grounding pad comprises a plurality of grounding pad sections, wherein each grounding pad section of the plurality of grounding pad sections is electrically isolated from every other grounding pad section.

14. The method of claim 11, further comprising affixing a fastener to the flexible body, wherein the fastener is configured to attach a first end of the flexible body to a second end of the flexible body.

15. The method of claim 11, further comprising providing a valve, wherein the valve is configured to put the inflatable bladder in a deflated state.

16. A system comprising:
a high frequency ablation device;
at least one electrode coupled to a high frequency generator of the high frequency ablation device, the at least one electrode configured to deliver high frequency ablation therapy to a patient; and
a grounding cuff, wherein the grounding cuff comprises:
an inflation bladder configured to be inflated with a fluid;
a grounding pad coupled to the inflation bladder and coupled to a first ground terminal, wherein a first surface of the grounding pad comprises an electrically conductive material and is configured to contact a skin of a patient;
one or more sensors positioned between a second surface of the grounding pad and the inflation bladder;
one or more sensors positioned between a second surface of the grounding pad and the inflation bladder, wherein the first ground terminal is distinct from a second ground terminal providing electrical grounding for the high frequency ablation device.

17. The system of claim 16, further comprising an insulating layer interposed between the second surface of the grounding pad and the one or more sensors, wherein the insulating layer comprises an electrically insulating material.

18. The system of claim 16, further comprising a data bus coupled to the grounding cuff and to the high frequency ablation device, wherein the one or more sensors are configured to gather first data corresponding to a position and an orientation of the grounding pad relative to the patient's skin and second data corresponding to a physiological state of the patient, and wherein the first data and the second data are sent to one or more processors of the high frequency ablation device via the data bus.

19. The system of claim 18, wherein the one or more processors are configured to:
determine, based on a metric indicative of a grounding provided by the grounding pad, that contact between the grounding pad and the skin of the patient is insufficient; and
send a control signal to cause the high frequency ablation device to cease delivering voltage to the one or more electrodes in response to a determination that the contact between the grounding pad and the skin of the patient is insufficient.

20. The system of claim 16, wherein the high frequency ablation device comprises one or more processors, and wherein the one or more processors are configured to adjust an output voltage of a high frequency ablation generator of the high frequency ablation device in response to receipt of data at a communication interface of the high frequency ablation device.
